SEQUENCE LISTING (5 sequences)

SEQ ID NO: 1 - DNA, 25 bp, Artificial Sequence
  antisense oligonucleotide
ttccagccagaaacctttacctgct

SEQ ID NO: 2 - DNA, 64 bp, Artificial Sequence
  Synthetic Sequence
acagcaggtaaaggtttctggctggaatgtcctgatctctggccaccctgctgtaaagtgctgg

SEQ ID NO: 3 - protein, 20 aa, Artificial Sequence
  Synthetic Sequence
QQIKVSGWNVLISGHPALDD

SEQ ID NO: 4 - DNA, 61 bp, Artificial Sequence
  Synthetic Sequence
acagcaggtaaaggtttctggctggaatgtcctgatctctggccaccctgctctcgatgac

SEQ ID NO: 5 - DNA, 140 bp, Artificial Sequence
  Synthetic Sequence
tctgatggttttgtcctcatccctgacctcaagtggaaccaacagcaggtaaaggtttctggctggaatgtcctgatctctggccaccctgctgtaagtgctggtccttctgactgccctctttctcacgctggcctgtc

(19) United States
(12) Patent Application Publication
(10) Pub. No.: US 2017/0355956 A1
(43) Pub. Date: Dec. 14, 2017

(54) COMPOSITIONS FOR INCREASING SURVIVAL OF MOTOR NEURON PROTEIN (SMN) LEVELS IN TARGET CELLS AND METHODS OF USE THEREOF FOR THE TREATMENT OF SPINAL MUSCULAR ATROPHY

(71) Applicant: RUTGERS, THE STATE UNIVERSITY OF NEW JERSEY, New Brunswick, NJ (US)

(72) Inventors: Megerditch KILEDJIAN, Whitehouse Station, NJ (US); Mi ZHOU, Chapel Hill, NC (US)

(21) Appl. No.: 15/506,917

(22) PCT Filed: Sep. 4, 2015
(86) PCT No.: PCT/US2015/048508

§ 371 Date: Feb. 27, 2017

Related U.S. Application Data
(60) Provisional application No. 62/045,911, filed on Sep. 4, 2014.

(51) Int. Cl. C12N 5/0793; A61K 38/46; A61K 48/00

(52) U.S. Cl. CPC C12N 5/0619; C12Y 306/01059; A61K 48/005; A61K 38/46; G01N 2800/28; G01N 2500/10; C12N 2503/04; C12N 2510/00; G01N 2333/914

(57) ABSTRACT

Compositions for altering splicing activity of the DcpS gene and increasing SMN protein expression in target cells are provided. Also disclosed are methods of use of such compositions for the treatment of SMA.

This application claims priority to U.S. Provisional Application No. 62/045,911 filed Sep. 4, 2014, the entire disclosure being incorporated herein by reference as though set forth in full.

Pursuant to 35 U.S.C. §202(c) it is acknowledged that the U.S. Government has rights in the invention described, which was made with funds from the National Institutes of Health, Grant No. GM067005.

### FIELD OF THE INVENTION

This invention relates to the fields of neuromuscular disorders and spinal muscular atrophy (SMA), in particular. More specifically, the invention provides compositions and methods which modulate survival of motor neuron (SMN) gene expression in target cells, thereby providing a new therapy for treatment of SMA.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

Spinal muscular atrophy (SMA) is an autosomal recessive neuromuscular disorder that results in symmetric proximal muscle weakness due to degeneration of motor neurons in the spinal cord (Lorson et al., 1999). The SMA phenotype is associated with expression levels of two genes, survival of motor neuron 1 (SMN1) and SMN2, with the phenotype resulting from homozygous mutations in SMN1. The two genes differ primarily by a translationally silent mutation in SMN2 that disrupts splicing of exon 7 leading predominantly to exon 7 exclusion and the generation of nonfunctional truncated protein (FIG. 1) (Garilov et al., 1998; Monani et al., 1999). However, due to a low level of exon 7 inclusion, SMN2 is a modifier gene for SMA severity and an increase in SMN2 mRNA levels can lead to dosage dependent compensation of SMN1 loss and a decrease of SMA severity in humans as well as mice model systems.

Despite years of research efforts, effective therapies for SMA are not yet available. Clearly, a need exists for the development of such therapies.

### SUMMARY OF THE INVENTION

In accordance with the present invention, a composition and method of use thereof comprising at least one agent that increases SMN protein expression levels in target cells of interest are disclosed. In one embodiment, the composition comprises a DcpS gene variant (DcpS^{In15}) encoded by a recombinant vector that increases SMN protein expression. In another embodiment, the composition comprises at least one vivo-morpholino oligonucleotide corresponding to sequence shown in FIG. 6 that modifies DcpS splicing activity to generate the DcpS^{In15} gene variant. The oligo in FIG. 6 has an antisense oligonucleotide splice switching mechanism. In an additional embodiment, the composition comprises at least one small molecule that modifies DcpS splicing activity to generate the desired DcpS^{In15} gene variant.

Also provided is a method for increasing SMN protein expression in a cell or tissue comprising: contacting said cells or tissue with an effective amount of at least one agent as described above which modifies DcpS splicing activity resulting in an increase in expression of the DcpS^{In15} variant thereby increasing SMN2 expression relative to untreated cells. In one embodiment, the agent is a peptide (or polypeptide) corresponding to the intact DcpS^{In15} protein or functional fragments thereof which are effective to increase SMN. In another embodiment, the agent is a nucleic acid molecule selected from dsRNA, antisense oligonucleotides and/or vectors encoding the same. Such agents are effective to modulate SMN-dependent activities, for example, enhancing survival of motor neurons. The aforementioned method is useful for the treatment or management of Spinal Muscular Atrophy and the alleviation of symptoms associated with SMA.

### BRIEF DESCRIPTION OF THE DRAWINGS

FIG. 1. Splice patterns of the SMN1 and SMN2 genes are shown. Spinal Muscular Atrophy (SMA) results from mutations in the Survival Motor Neuron (SMN) 1 gene leading to the loss of SMN1 expression, which can partially be compensated by the SMN2 gene. The splicing pattern of the SMN2 gene predominantly results in exon 7 skipping and translation of a nonfunctional protein. However, ˜10% of SMN2 pre-mRNA is spliced with the inclusion of exon 7 to generate low levels of functional SMN protein.

FIG. 2. DcpS^{In15} increases SMN2 mRNA. Full-length SMN2 mRNA levels were determined by qRT-PCR using 5′ primers ending with the C→T transition to distinguish between SMN1 and 2. Shown are wild type (+1+), heterozygote (+/In15), two different homozygous (In15/In15) cells (described in Ahmed et al., 2015, Hum Mol Gen) and the data presented relative to levels in wild type cells set to 1. SMN2, mRNA levels increase in the homozygous cells. Average of three biological replicates.

FIGS. 3A-3C. DcpS^{In15} increases fl-SMN2 mRNA and SMN protein in GM03813 SMA patient cells. FIG. 3A. fl-SMN2 mRNA levels were tested in GM03813 cells knocked down for DcpS and stably expressing the indicated shRNA-resistant DcpS variants. DcpS^{In15}, but not the catalytically inactive mutant DcpS^{MT} protein, increases fl-SMN2. (Average of three biological replicates) FIG. 3B. Western of SMN and DcpS proteins from cells used in A. (Average of two biological replicates). FIG. 3C. SMN protein levels increase in GM0813 cells expressing DcpS^{In15} with a background of endogenous levels of DcpS protein.

FIG. 4A-4B. SMN levels increase in cells expressing DcpS^{In15} at levels at least equivalent to or greater than endogenous DcpS^{WT}. FIG. 4A. Western blot of GM00323 cells expressing increasing amounts of DcpS^{In15} with corresponding increase of SMN protein shown. FIG. 4B. Quantitation of SMN protein levels in DcpS^{In15}/DcpS^{WT} ratio of ≦0.25 where there is no elevation of SMN protein, and ≧1 where SMN protein is increased. (Average of three biological replicates and titration.

FIG. 5 shows the results from assays which reveal loss of decapping activity by the recombinant DcpS^{In15} protein.

FIG. 6. Splice switching of endogenous DcpS intron 4 to the DcpS^{In15} alternative site. Vivo-morpholino antisense oligonucleotide (red line) was added to GM03813 cells and DcpS splicing tested. Schematic on lower right depicts the splicing pattern resulting with the indicated concentration of vivo-morpholino and arrows denote the primers used. Endogenous splicing is inhibited and shifted to the alternative site 45nt downstream as well as exon 4 skipping. Bottom panel is a control of exon2-exon3 junction not affected by the oligonucleotide. An exemplary antisense molecule comprises 5′ TTCCAGCCAGAAA CCTTTACCTGCT 3′ (SEQ ID NO: 1). However, antisense oligonucleotides can be designed to hybridize to the any contiguous 20-25 mer present in the DcpS variant sequence shown in FIG. 6.

### DETAILED DESCRIPTION OF THE INVENTION

Spinal muscular atrophy (SMA) is an autosomal recessive disease caused by a genetic mutation of the SMN1 and SMN2 genes, which encodes SMN. Survival of motor neurons are dependent on SMN protein expression as decreased levels of the protein results in death of neuronal cells in the anterior horn of the spinal cord and subsequent system-wide muscle wasting (atrophy) and infantile death (Lefebvre et al., 1995).

Scavenger decapping enzyme (DcpS) has been identified as a therapeutic target for Spinal Muscular Atrophy (Singh et al., 2008). DcpS is part of the RNA degradation machinery. Inhibition of DcpS may help to reduce the efficiency of mRNA turnover. DcpS functions in the last step of mRNA decay to hydrolyze the cap structure (m⁷GpppN) following 3′ to 5′ exonucleolytic decay to generate m⁷Gp+ppN products. As such, DcpS is a modulator of cap dinucleotide and m⁷GMP levels in cells and could indirectly affect downstream functions by controlling methylated nucleotide levels. Disruption of the yeast DcpS homolog, Dcs1, results in accumulation of cap dinucleotide and inhibition of 5′ to 3′ exonuclease activity.

The instant invention encompasses compositions and methods effective to increase SMN protein expression, providing a therapy for SMA patients. A DcpS variant (DcpS^{In15}), which results from an insertion of 15 amino acids into the protein, was identified which significantly increase SMN2 and SMN protein expression in SMA patient cells. The DcpS^{In15} variant acts as a gain of function protein, and acts to modify the SMA phenotype. Utilizing antisense oligonucleotide splice switching to modify the splicing pattern of the endogenous DcpS gene to the DcpS^{In15} variant in 293T cells, an increased level of DcpS^{In15} mRNA was observed. These data indicate that shifting of the endogenous DcpS gene to express the DcpS^{In15} variant can be accomplished with a vivo-morpholino oligonucleotide which enhances SMN protein expression, thereby providing a therapeutic benefit for SMA patients.

### Definitions

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“A DcpS nucleic acid” as used herein refers to a scavenger decapping enzyme (DcpS) encoding nucleic acid. The nucleic acid molecule may be isolated from a natural source by cDNA cloning or subtractive hybridization or synthesized manually. The nucleic acid molecule may be synthesized manually by the triester synthetic method or by using an automated DNA synthesizer. The DcpS nucleic acid can be targeted for alternative splicing by the agents of the present invention to generate the DcpS^{In15} variant disclosed herein.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to the target nucleic acid, but does not hybridize to other nucleotides. Also polynucleotide which “specifically hybridizes” may hybridize only to a target DcpS gene sequence. Appropriate conditions enabling specific hybridization of single stranded nucleic acid molecules of varying complementarity are well known in the art.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the DcpS nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the nucleic acid molecule of interest. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein or functional peptide fragment thereof produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which is obtained from a test subject or patient. Samples may include but are not limited to cells, body fluids, including blood, serum, plasma, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of a DpcS gene variant or its encoded protein. Agents are evaluated for potential biological activity by inclusion in screening assays described hereinbelow.

### Kits and Articles of Manufacture

Any of the aforementioned products can be incorporated into a kit which may contain a DcpS variant polynucleotide or fragment thereof, a Gene Chip, an oligonucleotide which modulates splicing, a polypeptide or peptide which modulates splicing, an antibody, a label, marker, or reporter, a pharmaceutically acceptable carrier, a physiologically acceptable carrier, instructions for use, a container, a vessel for administration, an assay substrate, or any combination thereof.

### Methods of Using Cells Expressing DcpS and/or a Splice Variant Thereof for Development of Therapeutic Agents

Also provided in the present invention are methods for identifying agents which modulate the splicing activity of the DcpS gene for the amelioration of SMA. Specific organic molecules can thus be identified with capacity to alter the splicing pattern of the DcpS gene and its encoded product, thereby generating efficacious therapeutic agents for the treatment of a variety of SMA.

Having observed that a variant of DcpS gives rise to an altered splicing pattern resulting in increased expression of SMN, provides a new target for the rational design of therapeutic agents which modulate SMN levels in target cells. Small peptide molecules corresponding to these regions may be used to advantage in the design of therapeutic agents which effectively modulate the activity, or alter the expression level of proteins of interest.

Molecular modeling should facilitate the identification of specific organic molecules with capacity to modulate splicing based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered DpcS gene or DpcS gene variant. These host cell lines or cells are defective at the polypeptide level. The host cell lines or cells are grown in the presence of drug compound. SMN levels are then determined to assess if the compound is capable of regulating in increasing the expression level thereof in the defective cells. Host cells contemplated for use in the present invention include but are not limited to mammalian cells, fungal cells, insect cells, and neuronal cells. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the sequences of the present invention or functional fragments thereof provide a system in which to screen potential compounds or agents for the ability to modulate the expression levels of SMN. Thus, in one embodiment, the nucleic acid molecules of the invention may be used to create recombinant cell lines for use in assays to identify agents which modulate splicing of DcpS. Also provided herein are methods to screen for compounds capable of modulating the function of SMN proteins.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the DpcS nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of human DcpS containing nucleic acids enables the production of strains of laboratory mice carrying the human DcpS gene. Transgenic mice expressing the human DcpS gene of the invention provide a model system in which to examine the therapeutic effect of agents in enhancing SMN expression and ameliorating SMA. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in SMA. Such mice provide an in vivo screening tool to study putative thereapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use DcpS genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing DcpS nucleic acid as a targeted insertional cassette provides means to detect a successful insertion as visualized, for example, by acquisition of immunoreactivity to an antibody immunologically specific for the polypeptide encoded by the introduced nucleic acid and, therefore, facilitates screening/selection of ES cells with the desired genotype.

As used herein, a knock-in animal is one in which the endogenous murine gene, for example, has been replaced with human DcpS gene, gene fragment or gene variant such as DcpS^{In15}. Such knock-in animals provide an ideal model system for studying the development of SMA.

As used herein, the expression of a DcpS nucleic acid, fragment thereof, can be targeted in a “tissue specific manner” or “cell type specific manner” using a vector in which nucleic acid sequences encoding all or a portion of DcpS are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding DcpS or a variant thereof may be operably linked to a variety of different promoter sequences for expression in transgenic animals. Such promoters include, but are not limited to a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; and Glial fibrillar acidic protein (GFAP) promoter for the expression of transgenes in glial cells.

Methods of use for the transgenic mice of the invention are also provided herein. Such transgenic mice are useful, for example, to develop screening methods to screen therapeutic agents to identify those capable of modulating or inhibiting the development of SMA.

### Pharmaceuticals and Peptide Therapies

A. Variant DcpS Variant Polypeptides

In a preferred embodiment of the present invention, variant DcpS variant polypeptides may be administered to a patient via infusion in a biologically compatible carrier, preferably via intravenous injection. The variant DcpS variants of the invention may optionally be encapsulated into liposomes or mixed with other phospholipids or micelles to increase stability of the molecule. DcpS variant may be administered alone or in combination with other agents known to alleviate symptoms of SMA. These include, without limitation, muscle relaxants such as baclofen, tizanidine, and the benzodiazepines which can reduce spasticity. Botulinum toxin may be used to treat jaw spasms or drooling. Excessive saliva can be treated with amitriptyline, glycopyolate, and atropine or by botulinum injections into the salivary glands. Antidepressants may be also be employed for treating depression which is often seen in SMA patients. An appropriate composition in which to deliver DcpS variant polypeptides may be determined by a medical practitioner upon consideration of a variety of physiological variables, including, but not limited to, the patient's condition and state of atrophy. A variety of compositions well suited for different applications and routes of administration are well known in the art and are described hereinbelow.

The preparation containing the purified DcpS^{In15} variant contains a physiologically acceptable matrix and is preferably formulated as a pharmaceutical preparation. The preparation can be formulated using substantially known prior art methods, it can be mixed with a buffer containing salts, such as NaCl, CaCl₂, and amino acids, such as glycine and/or lysine, and in a pH range from 6 to 8. Until needed, the purified preparation containing the variant can be stored in the form of a finished solution or in lyophilized or deep-frozen form. Preferably the preparation is stored in lyophilized form and is dissolved into a visually clear solution using an appropriate reconstitution solution.

Alternatively, the preparation according to the present invention can also be made available as a liquid preparation or as a liquid that is deep-frozen.

The preparation according to the present invention is especially stable, i.e., it can be allowed to stand in dissolved form for a prolonged time prior to application.

The preparation according to the present invention contains a DcpS variant or DcpS targeted small molecule which is able to elevate SMN2 mRNA and SMN protein levels in SMA patient cells.

Prior to processing the purified protein or small molecule (e.g., siRNA, antisense olignucleotide etc.) into a pharmaceutical preparation, the purified protein or small molecule is subjected to the conventional quality controls and fashioned into a therapeutic form of presentation. In particular, during the recombinant manufacture, the purified preparation is tested for the absence of cellular nucleic acids as well as nucleic acids that are derived from the expression vector, preferably using a method, such as is described in EP 0 714 987.

The pharmaceutical preparation may contain dosages of between 10-1000 μg/kg, more preferably between about 10-250 μg/kg and most preferably between 10 and 75 μg/kg. Patients may be treated immediately upon presentation at the clinic with SMA. Alternatively, patients may receive a bolus infusion as needed.

B. DcpS Variant-Encoding Nucleic Acids

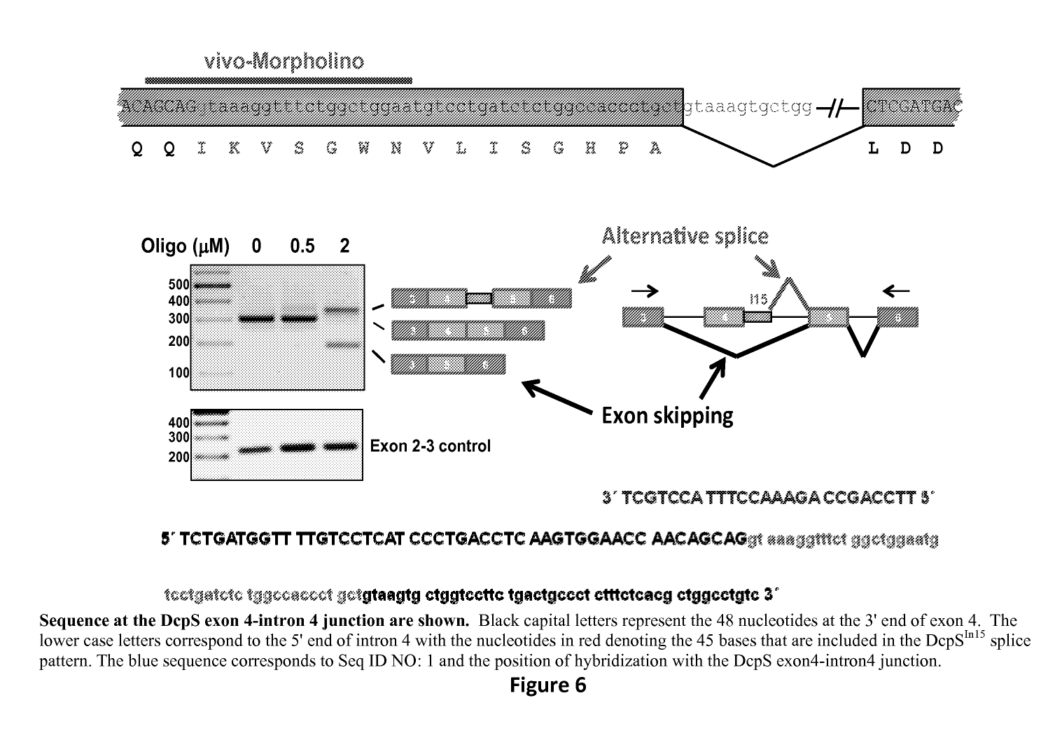

DcpS variant-encoding nucleic acids may be used for a variety of purposes in accordance with the present invention. In a preferred embodiment of the invention, a nucleic acid delivery vehicle (i.e., an expression vector) for modulating the SMA phenotype is provided wherein the expression vector comprises a nucleic acid sequence coding for the antisense oligonucleotide shown in FIG. 6 for example. Administration of such expression vectors to a patient results in increased levels of SMN production which serves to alter the SMA phenotype. In accordance with the present invention, an DcpS variant encoding nucleic acid sequence may encode an DcpS variant polypeptide as described herein whose expression reduces the SMA phenotype.

Expression vectors comprising variant DcpS sequences or olignucleotides targeting DcpS may be administered alone, or in combination with other molecules useful for modulating SMA phenotype. According to the present invention, the expression vectors or combination of therapeutic agents may be administered to the patient alone or in a pharmaceutically acceptable or biologically compatible compositions.

In a preferred embodiment of the invention, the expression vector comprising nucleic acid sequences encoding the variant DcpS or targeting DcpS is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors.

In a preferred embodiment of the present invention, methods are provided for the administration of a viral vector comprising nucleic acid sequences encoding a variant DcpS variant, or a small nucleic acid molecule directed thereto. Adenoviral vectors of utility in the methods of the present invention preferably include at least the essential parts of adenoviral vector DNA. As described herein, expression of a variant DcpS variant polypeptide following administration of such an adenoviral vector serves to modulate SMA phenotype.

Recombinant adenoviral vectors have found broad utility for a variety of gene therapy applications. Their utility for such applications is due largely to the high efficiency of in vivo gene transfer achieved in a variety of organ contexts.

Adenoviral particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, adenoviruses are known to infect a wide variety of cell types in vivo and in vitro by receptor-mediated endocytosis. Attesting to the overall safety of adenoviral vectors, infection with adenovirus leads to a minimal disease state in humans comprising mild flu-like symptoms.

Due to their large size (˜36 kilobases), adenoviral genomes are well suited for use as gene therapy vehicles because they can accommodate the insertion of foreign DNA following the removal of adenoviral genes essential for replication and nonessential regions. Such substitutions render the viral vector impaired with regard to replicative functions and infectivity. Of note, adenoviruses have been used as vectors for gene therapy and for expression of heterologous genes.

For a more detailed discussion of the use of adenovirus vectors utilized for gene therapy, see Berkner, 1988, Biotechniques 6:616-629 and Trapnell, 1993, Advanced Drug Delivery Reviews 12:185-199.

It is desirable to introduce a vector that can provide, for example, multiple copies of a desired gene and hence greater amounts of the product of that gene. Improved adenoviral vectors and methods for producing these vectors have been described in detail in a number of references, patents, and patent applications, including: Mitani and Kubo (2002, Curr Gene Ther. 2(2):135-44); Olmsted-Davis et al. (2002, Hum Gene Ther. 13(11):1337-47); Reynolds et al. (2001, Nat Biotechnol. 19(9):838-42); U.S. Pat. No. 5,998,205 (wherein tumor-specific replicating vectors comprising multiple DNA copies are provided); U.S. Pat. No. 6,228,646 (wherein helper-free, totally defective adenovirus vectors are described); U.S. Pat. No. 6,093,699 (wherein vectors and methods for gene therapy are provided); U.S. Pat. No. 6,100,242 (wherein a transgene-inserted replication defective adenovirus vector was used effectively in in vivo gene therapy of peripheral vascular disease and heart disease); and International Patent Application Nos. WO 94/17810 and WO 94/23744.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for the expression of the variant DcpS variants or functional fragments thereof. For example, an E1 deleted type 5 adenoviral vector comprising nucleic acid sequences encoding variant DcpS variant under the control of a cytomegalovirus (CMV) promoter may be used to advantage in the methods of the present invention.

Also included in the present invention is a method for modulating SMA phenotype comprising providing cells of an individual with a nucleic acid delivery vehicle encoding a variant DcpS variant polypeptide and allowing the cells to grow under conditions wherein the DcpS variant polypeptide is expressed.

From the foregoing discussion, it can be seen that DcpS variant polypeptides, and DcpS variant polypeptide expressing nucleic acid vectors may be used in the treatment of disorders associated with SMA.

C. Pharmaceutical Compositions

The expression vectors of the present invention may be incorporated into pharmaceutical compositions that may be delivered to a subject, so as to allow production of a biologically active protein or small molecule (e.g., a variant DcpS variant polypeptide or functional antisense oligonucleotide or dsRNA). In a particular embodiment of the present invention, pharmaceutical compositions comprising sufficient genetic material to enable a recipient to produce a therapeutically effective amount of SMN protein thereby influencing the SMA phenotype in the subject. Alternatively, as discussed above, an effective amount of the variant DcpS^{In15} polypeptide may be directly infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., muscle relaxers, botulinum toxin) which modulate SMA symptoms.

In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of DcpS variant-containing vectors or polypeptides, such labeling would include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques and guidance provided in the present invention. Therapeutic doses will depend on, among other factors, the age and general condition of the subject, the severity of the SMA phenotype, and the strength of the control sequences regulating the expression levels of the variant DcpS variant polypeptide. Thus, a therapeutically effective amount in humans will fall in a relatively broad range that may be determined by a medical practitioner based on the response of an individual patient to vector-based DcpS variant treatment.

D. Administration

The variant DcpS polypeptides, alone or in combination with other agents may be directly infused into a patient in an appropriate biological carrier as described hereinabove. Expression vectors of the present invention comprising nucleic acid sequences encoding variant DcpS variant, or functional fragments thereof, may be administered to a patient by a variety of means (see below) to achieve and maintain a prophylactically and/or therapeutically effective level of the DcpS variant polypeptide. One of skill in the art could readily determine specific protocols for using the DcpS variant encoding expression vectors of the present invention for the therapeutic treatment of a particular patient. Protocols for the generation of adenoviral vectors and administration to patients have been described in U.S. Pat. Nos. 5,998,205; 6,228,646; 6,093,699; 6,100,242; and International Patent Application Nos. WO 94/17810 and WO 94/23744, which are incorporated herein by reference in their entirety.

Variant DcpS variant encoding adenoviral vectors of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. A clinician specializing in the treatment of patients with SMA may determine the optimal route for administration of the adenoviral vectors comprising DcpS variant nucleic acid sequences based on a number of criteria, including, but not limited to: the condition of the patient and the purpose of the treatment (e.g., alleviation of SMA symptoms).

The present invention also encompasses AAV vectors comprising a nucleic acid sequence encoding a variant DcpS variant polypeptide.

Also provided are lentivirus or pseudo-typed lentivirus vectors comprising a nucleic acid sequence encoding a variant DcpS variant polypeptide

Also encompassed are naked plasmid or expression vectors comprising a nucleic acid sequence encoding a variant DcpS variant polypeptide.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following example is provided to illustrate certain embodiments of the invention. It is not intended to limit the invention in any way.

### Example I

### A Variant of DcpS is Effective to Increase SMN Protein Levels in SMA Cells

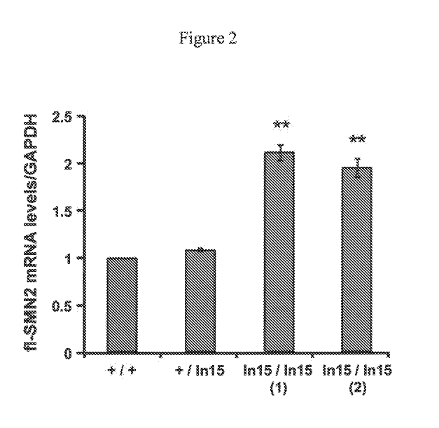

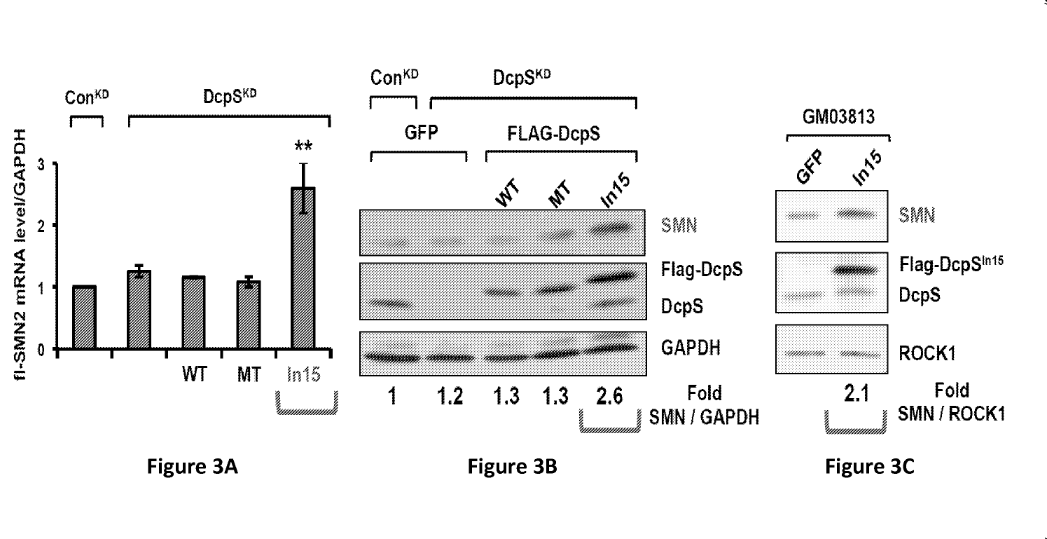

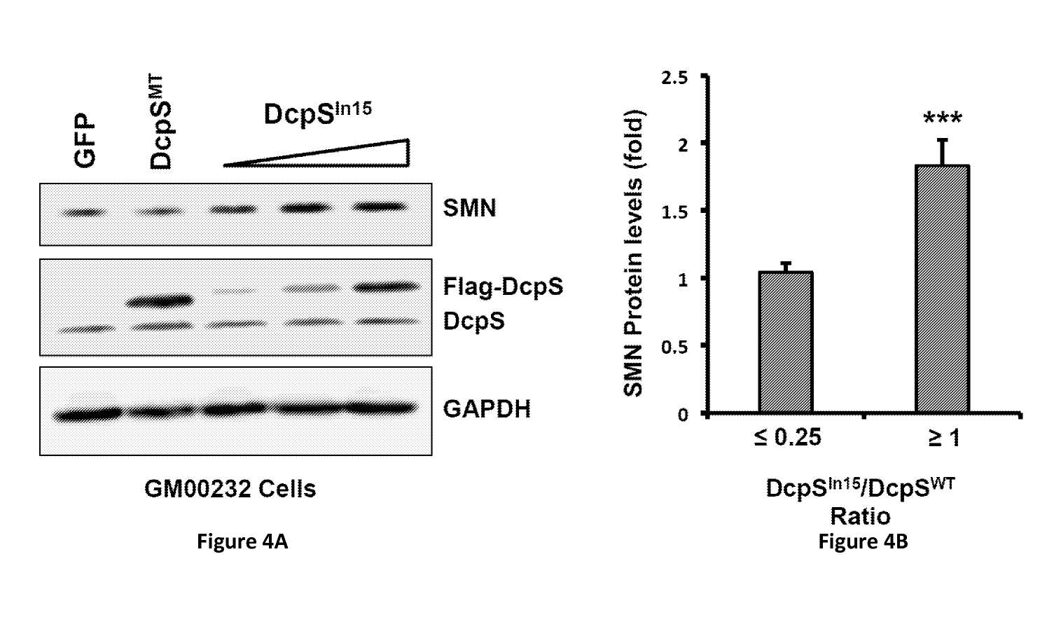

We now have evidence that a variant form of DcpS, initially identified as a mutation in non-syndromic autosomal recessive intellectual disability (ID) and results in the insertion of an additional 15 amino acids into the protein (DcpS^{In15}), can significantly elevate SMN2 mRNA and SMN protein in SMA patient cells. Cells containing a homozygous DcpS^{In15} allele exhibit a 2 fold increase in SMN2 mRNA levels (FIG. 2). More significantly, exogenous expression of DcpS^{In15} into SMA patient fibroblast cells within a background of reduced endogenous DcpS, resulted in the elevation of both SMN2 mRNA and protein (FIG. 3). The same is not observed when the cells are complemented with either wild type or a catalytically inactive DcpS (FIGS. 3A and 3B). This latter point is significant because it indicates SMN2 elevation is not a consequence of inhibiting DcpS decapping activity per se. Furthermore, SMN levels increase in cells expressing DcpS^{In15} to levels equivalent or greater than endogenous DcpS^{WT} (FIGS. 4A and 4B). These data demonstrate that the DcpS^{In15} variant is a gain of function protein and DcpS^{In15} is effective to modify the SMA phenotype.

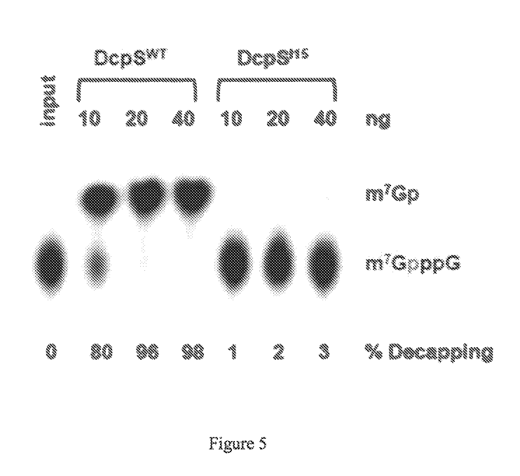

The variant DcpS results from a single nucleotide mutation at the endogenous DcpS intron 4 splice site that leads to the utilization of a alternative splice site 45 nucleotides downstream and results in the insertion of 15 amino acids into the DcpS protein (DcpS^{In15}). The resulting DcpS^{In15} protein loses decapping activity and is unable to hydrolyze cap structure (FIG. 5).

Shifting of the endogenous DcpS gene to express the DcpS^{In15} variant can also be accomplished by antisense oligonucleotide splice switching technology. With this approach, an antisense oligonucleotide complementary to the endogenous splice site is used to block the endogenous site and shift the splicing pattern to the DcpS^{In15} pattern in SMA patient cells. The objective is to alter the endogenous DcpS splicing in order to generate sufficient levels of DcpS^{In15} thereby elevating SMN2 gene expression and increasing levels of full length SMN protein. 293T cells were treated with 5 μM antisense morpholino oligonucleotide containing a cell permeable guanidinium based dendramer delivery system (vivo-morpholino; GeneTools LLC) for 24 hours and splicing patterns determined by RT-PCR. As shown in FIG. 6, the addition of the vivo-morpholino oligonucleotide completely inhibits utilization of the endogenous exon 4 splice site. Instead ˜50% of the splicing utilizes the DcpS^{In15} splice site 45 nucleotides downstream analogous to that observed in the homozygous DcpS^{In15} ID patient cells, and ˜50% results in both sites being blocked and skipping of exon 4 entirely. In the latter case (lower panel in FIG. 6), splicing bypasses exon 4 and goes from exon 3 to exon 5. These data clearly demonstrate that DcpS splicing can be shifted to the DcpS^{In15} variant pattern in cells. The invention also encompasses optimized oligonucleotide sequences comprising altered nucleotide backbones, and small molecules that can shift splicing to further improve levels of DcpS^{In15}, thereby elevating SMN2 levels and providing a therapeutic intervention for SMA patients.

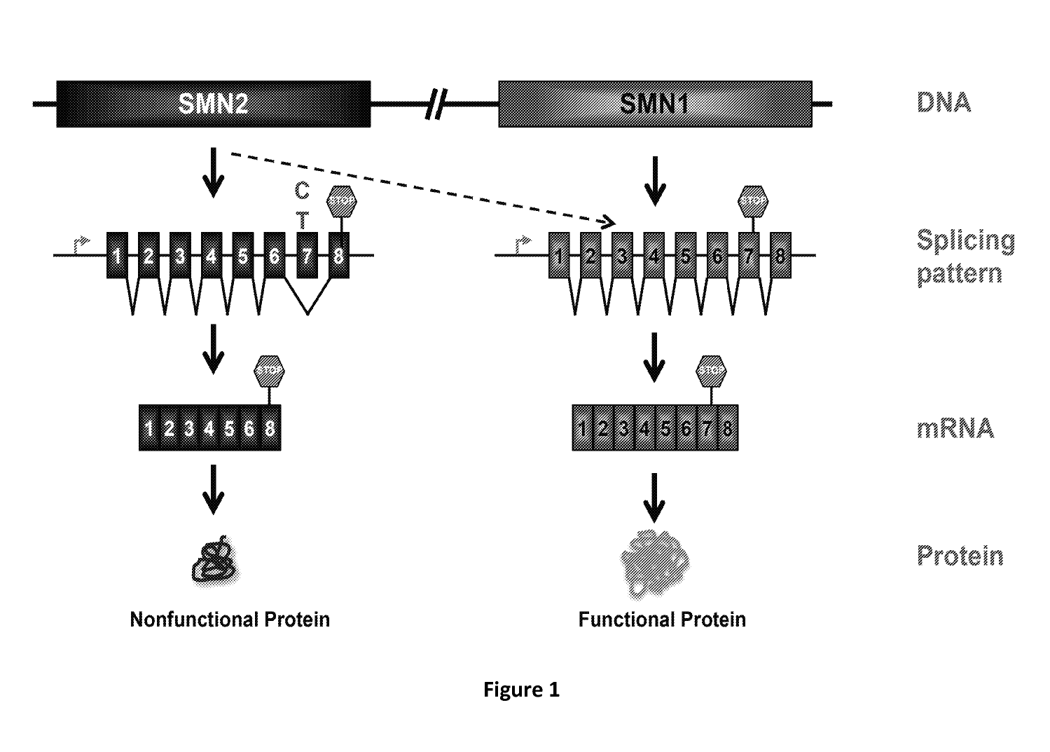

Additional measures that could be envisioned to increase the level of DcpS^{In15} variant protein in cells could be through enhanced translation or decreased decay of the DcpS^{In15} protein. Despite similar levels of wild type and variant DcpS mRNA in the +/In15 individual in FIG. 1, DcpS^{In15} protein levels are one quarter of that of wild type protein. If the reduced levels of DcpS^{In15} protein are a function of reduced stability, the potential exists to incorporate current FDA approved proteasome or lysosomal protein degradation inhibitors (carfilzomib and chloroquine respectively) to increase protein levels for therapeutic applications.

### REFERENCES

Lorson, C. L., Hahnen, E., Androphy, E. J., and Wirth, B. (1999). A single nucleotide in the SMN gene regulates splicing and is responsible for spinal muscular atrophy. Proc Natl Acad Sci USA 96, 6307-6311.
Monani, U. R., Lorson, C. L., Parsons, D. W., Prior, T. W., Androphy, E. J., Burghes, A. H., and McPherson, J. D. (1999). A single nucleotide difference that alters splicing patterns distinguishes the SMA gene SMN1 from the copy gene SMN2. Hum Mol Genet 8, 1177-1183.
Gavrilov, D. K., Shi, X., Das, K., Gilliam, T. C., and Wang, C. H. (1998). Differential SMN2 expression associated with SMA severity. Nat Genet 20, 230-231.
Lefebvre, S., Burglen, L., Reboullet, S., Clermont, O., Burlet, P., Viollet, L., Benichou, B., Cruaud, C., Millasseau, P., and Zeviani, M., 1995). Identification and characterization of a spinal muscular atrophy-determining gene. Cell 80, 155-165.
Singh, J., Salcius, M., Liu, S. W., Staker, B. L., Mishra, R., Thurmond, J., Michaud, G., Mattoon, D. R., Printen, J., and Christensen, J., (2008). DcpS as a therapeutic target for spinal muscular atrophy. ACS Chem Biol 3, 711-722.
Ausebel et al. eds., Current Protocols in Molecular Biology, John Wiley & Sons, NY, N.Y. 1995.
Sambrook et al. Molecular Cloning: A Laboratory Manuel or Current Protocols in Molecular Biology 16.3-17.44 (1989).
Evans, M. J. and Kaufman, M. H. (1981). Establishment in culture of pluriopotential cells from mouse embryos. Nature 292, 154-156.
Gossler, A., Korn, R., Serfling, E., and Kemler, R. (1984). Transgenesis by means of blastocyst-derived embryonic stem cell lines. Proc. Natl. Acad. Sci. 83, 9065-9069.

What is claimed is:

1. A method for screening agents which increase production of a DcpS variant in a cell for increasing production of survivor motor neuron (SMN) protein in spinal muscular atrophy (SMA) cells, comprising
a) providing cells expressing the DcpS gene;
b) incubating said cells in the presence and absence of an agent that modifies DcpS gene splicing pattern in order to increase production of a DcpS^{In15} variant; and
d) identifying agents which increase production of said DcpS^{In15} variant, said increase in said variant causing an increase in survivor motor neuron (SMN) protein relative to untreated control cells.

2. The method of claim 1, wherein said cells are SMA fibroblast cells.

3. The method of claim 1, wherein said cells within a tissue.

4. The method of claim 1, wherein said cells are mammalian in origin.

5. The method of claim 3, wherein said tissues are mammalian in origin.

6. The method of claim 1, wherein said cells are in a mouse model of SMA.

7. The method of claim 3, wherein said tissues are in a mouse model of SMA.

8. The method of claim 1 wherein said cells are transformed with a naked DNA encoding said DcpS^{In15} variant sequence.

9. The method of claim 1, wherein said cells are transformed with a vector encoding said DcpS^{In15} variant sequence.

10. The method of claim 9, wherein said cells are transformed with said vector selected from retroviruses, adeno-associated viruses, herpesviruses or adenoviruses expressing a DcpS^{In15} variant sequence or an oligonucleotide which alters the splicing pattern of a DcpS^{In15} sequence.

11. The method of claim 1, wherein said agent is selected from the group consisting of polypeptides, peptides, peptoids, small inorganic molecules, small organic molecules, nucleic acids, anti-sense nucleic acids, oligonucleotides, synthetic oligonucleotides, and carbohydrates,

12. The method of claim 1, wherein said agent is an anti-sense vivo-morpholino.

13. An agent identified by the method of claim 1.

14. A method for treatment or management of SMA in a patient in need thereof, comprising administration of an effective amount of an agent which increases survival motor neuron protein (SMN) levels in cells from said patient, thereby alleviating or modulating SMA symptoms in said patient.

15. The method of claim 14, wherein said agent is a DcpS^{In15} variant protein.

16. The method of claim 15, wherein said agent is infused into a patient in a pharmaceutically acceptable carrier.

17. The method of claim 14, wherein said agent is an antisense oligonucleotide of SEQ ID NO: 1.

18. The method of claim 14, wherein said agent is a nucleic acid encoding DcpS^{In15} variant protein in an expression vector.

19. The method of claim 14, wherein said agent is an antisense oligonucleotide of SEQ ID NO: 1 cloned within an expression vector.
